(19) United States
(12) Patent Application Publication
(10) Pub. No.: US 2007/0120113 A1
(43) Pub. Date: May 31, 2007

(54) Supramolecular structures and method for forming the same

(76) Inventors: King-Fu Lin, Taipei (TW); Chi-Chun Hsieh, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/287,309

(22) Filed: Nov. 28, 2005

(51) Int. Cl. H01L 51/00; C07D 495/02; C07D 409/14

(52) U.S. Cl. 257/40; 549/50; 549/59; 558/406; 977/700

(57) ABSTRACT

A primary supramolecular structure is described. The primary supramolecular structure has a shape of ring-like disk. The shape of ring-like disk has a diameter of about 10 nanometers to about 60 nanometers. The mentioned primary supramolecular structure is formed by self-assembly of amphiphilic conjugate molecules. Moreover, a secondary supramolecular structure is described. The secondary supramolecular structure has a shape of ring-like disk. The shape of ring-like disk has a diameter of about 100 nanometers to about 300 nanometers. The mentioned secondary supramolecular structure is formed by self-assembly of amphiphilic conjugate molecules hybrid with metal alkoxides or non-metal alkoxides.

### BACKGROUND OF INVENTION

1. Field of the Invention

The present invention relates to supramolecular structure. More specifically, the present invention relates to a supramolecular structure and a method for forming the supramolecular structure through amphiphilic conjugate molecules.

2. Description of the Related Art

Supramolecular architectures of π-conjugated molecules have attracted great attention during the past decade because of their potential applications on the optoelectronic nanodevices. It is a commonly accepted concept that the optoelectronic properties of π-conjugated molecules are affected not only by the primary molecular structure (π-conjugation) but by the supramolecular organization (π-stacking) similar to that of the proteins and block copolymers. For example, Stupp et al. have found that the self-assembly of the conjugated molecules into supramolecular ribbon nanostructures leads to enhanced electronic conductivity due to improved π-stacking region. To this end, amphiphilic molecules with a proper hydrophilicity or hydrophobility are needed to proceed a self-assembly process. Researches in the field of self-assembly techniques usually focus first on the design and synthesis of self-assembling amphiphilic or liquid-crystalline molecules, followed by the investigation of aggregation behavior in liquid state as well as in solid state.

On the other hand, since the discovery of the M41's family of silicate mesoporous molecular sieves by Mobil researchers in 1992, surfactant-templated silica or titania with ordered nanostructures becomes a promising candidate for optoelectronic devices such as solar cells. Considering that the amphiphilic molecule itself can also play the role of surfactant, it may be used as a template to construct the hybrid nanostructures with nanopatterns that have a potential application on the nano-sized devices.

Accordingly, there is still a need to synthesize new amphiphilic conjugate molecules. The new amphiphilic conjugate molecules are synthesized to have substantially the same sizes, good stabilities and photoelectric efficiencies. There is also a need to provide a simpler method for synthesizing amphiphilic conjugate molecules, so as to meet the industrial requirements.

### SUMMARY OF THE INVENTION

According to the description of the related art, to meet the industrial requirements, the invention provides a supramolecular structure, and provides a method for forming the supramolecular structure through amphiphilic conjugate molecule.

According to the embodiments of the invention, an object of the invention is to provide an amphiphilic conjugate molecule. The amphiphilic conjugate molecule comprises a hydrophobic segment being π-conjugated, a hydrophilic segment and a linking group. The hydrophobic segment has at least two aromatic structures. The linking group links the hydrophobic segment with the hydrophilic segment. The linking group twists, to have a twist, between the linked hydrophobic segment and the hydrophilic segment of the amphiphilic conjugate molecule.

According to the embodiments of the invention, another object of the invention is to provide an amphiphilic conjugate molecule with a twist in between hydrophobic OPV segments and hydrophilic PEO segments render them to have only one side with the same curvature to proceed π-π stacking. Such packing under the same curvature favors formation of a shape of ring-like disk, so as to form the primary supramolecular structure.

It is further object of the invention to provide a secondary supramolecular structure. The secondary supramolecular structure, having a shape of ring-like disk, is formed of a plurality of metal or non-metal alkoxides and amphiphilic conjugate molecules. Therefore, the secondary supramolecular structure is a hybrid supramolecular structure. Additionally, the diameter of the secondary supramolecular structure is much larger than the diameter of the primary supramolecular structure. The sizes between different secondary supramolecular structures are more uniform than the sizes of the different primary supramolecular structures.

According to the above objects, the invention discloses two kinds of supramolecular structures formed by amphiphilic conjugate molecules. The primary supramolecular structure has a shape of ring-like disk. The shape of ring-like disk has a diameter of about 10 nanometers to about 60 nanometers. Additionally, the invention also discloses a secondary supramolecular structure (hybrid supramolecular structure) having a shape of ring-like disk. The secondary supramolecular structure is formed of a plurality of metal or non-metal alkoxides and amphiphilic conjugate molecules. The shape of ring-like disk has a diameter of about 100 nanometers to about 300 nanometers.

### BRIEF DESCRIPTION OF THE DRAWINGS

FIG. 1 is ¹H NMR spectrum of amphiphilic PEO₁₇—OPV₃ molecule;

FIG. 2 shows molecular graphics of amphiphilic PEO₁₇—OPV₃ using a Cerius 2 Energy Minimization (Red: oxygen atom; Gray: carbon atom; White; hydrogen atom; Yellow: sulfur atom);

FIG. 3 shows AFM images of amphiphilic PEO₁₇—OPV₃ deposited on a mica substrate. Top: Image size is 2×2 μm². Bottom: Enlarged image from the selection area of top image; and

FIG. 4 shows AFM images of PEO₁₇—OPV₃/silicate hybrid deposited on a mica substrate. Top: Image analysis of 1×1 μm² size. Bottom: Phase image.

### DESCRIPTION OF THE PREFERRED EMBODIMENTS

The invention directs to a method for forming a supramolecular structure through amphiphilic conjugate molecules. In order to thoroughly understand the invention, the detail process steps or the composition structure will be proposed as follows. Obviously, the execution of the invention is not limited to special detail techniques understood by one skilled in the organic synthesis domain. On the other hand, well known composition or the process steps are not described in detail, thereby preventing the invention from creating limits not necessity. In the following description, the preferred embodiments of the invention are described in detail. Besides the detailed description, however, the invention may also widely apply in other embodiments. The scope of the invention will be defined by the appended claims and not by the detailed description.

A first embodiment of the invention discloses an amphiphilic conjugate molecule comprising a hydrophobic segment being π-conjugated, a hydrophilic segment and a linking group. The hydrophobic segment comprises at least two aromatic structures. Specifically, the hydrophobic segment comprises one selected from the group consisting of 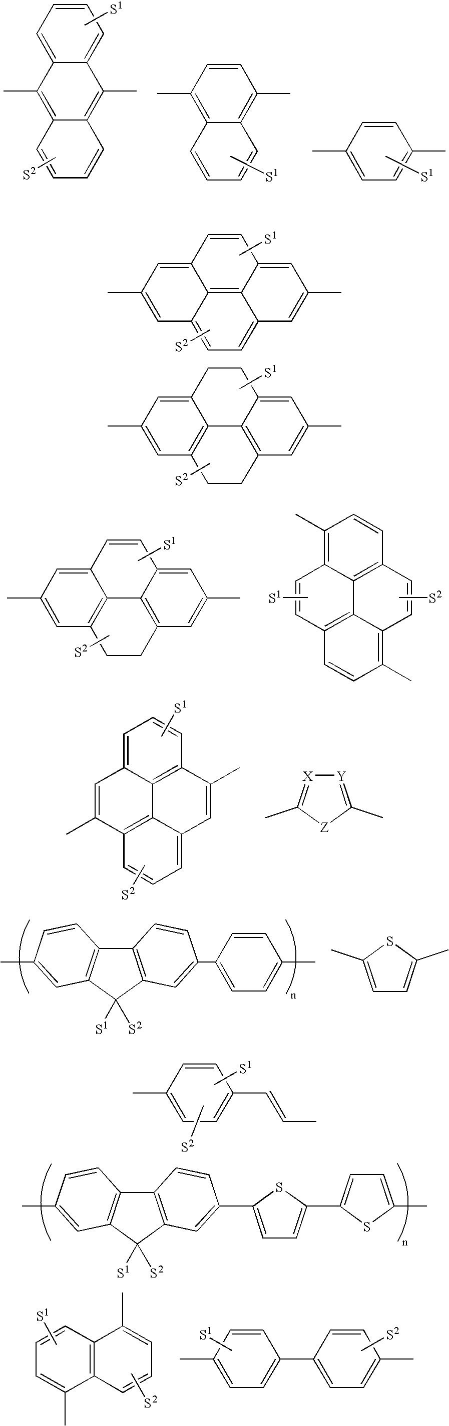 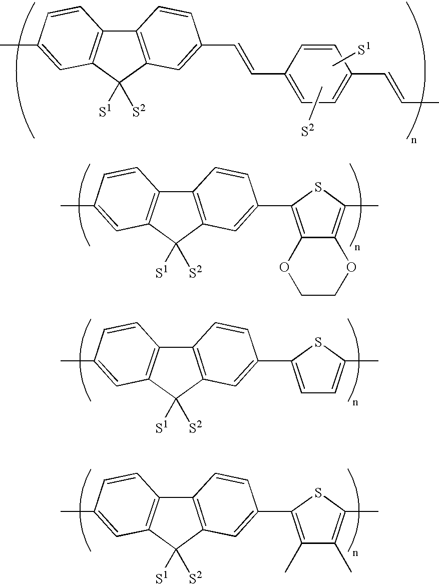
and the combination thereof, wherein two of the S¹, S², S³, S⁴ are identical or non-identical, and wherein the S¹, S², S³, S⁴ comprises one selected from the group consisting of a hydrogen atom, alkyl group, alkoxy group, cyclic alkyl group, aromatic group, heterocyclic group and the combination thereof. The X and Y are identical or non-identical, wherein the X and Y comprise one selected from the group consisting of CS1, N and the combination thereof. The Z one selected from the group consisting of —O—, —S—, —NS1-, —CS1S2-, —CS1=CS1-, —CS1=N— and the combination thereof. The Ph is a phenyl group. The Ar is an aromatic group. Additionally, the linking group links the hydrophobic segment with the hydrophilic segment, and twists between the linked hydrophobic segment and the hydrophilic segment of the amphiphilic conjugate molecule.

In the first embodiment, the hydrophilic segment comprises one selected from the group consisting of polyalkylene glycol, polyalkylene glycol monoalkyl ether and their derivatives, wherein monoalkyl is selected from the group consisting of CH₃, C₂H₅, C₃H₇, and C₄H₉. The polyalkylene glycol derivatives such as polyethylene glycol (PEG), polybutylene glycol (PBG), polypropylene glycol (PPG), PPG-PEG block or random polymers, and the preferred example of polyalkylene glycol monoalkyl ether is polyethylene glycol methyl ether. Additionally, the linking group comprises one selected from the group consisting of —CH₂—, —CR₂—and 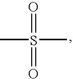
wherein the R is an alkyl group. On the other hand, in a preferred example, the amphiphilic conjugate molecule has the following structural formula: 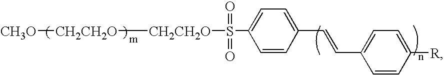
wherein m≧1, n≧1, and wherein the R is a cyclic alkyl group, a non-cyclic alkyl group, an aromatic group and a heterocyclic group.

### EXAMPLE 1

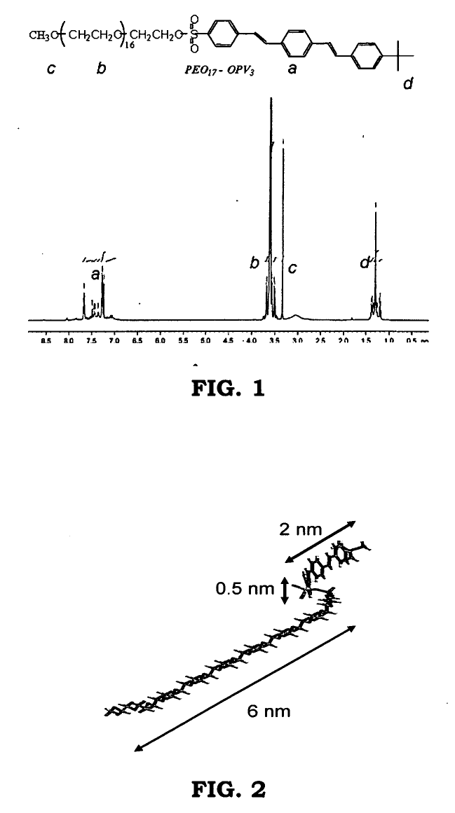

An amphiphilic conjugate molecule (abbreviated as “PEO₁₇—OPV₃”) having the following structural formula is provided: 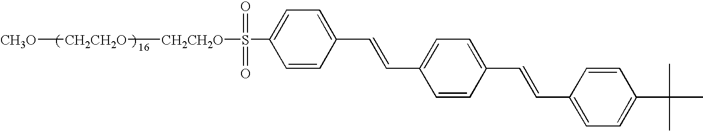
In the amphiphilic conjugate molecule, the PEO segment has hydrophilicity, whereas the OPV segment has hydrophobility. Referring to FIG. 1, there is no resonance peak with δ=6.5˜6.8 ppm in the ¹H-nuclear magnetic resonance spectrum. Accordingly, the PPV trimer may have co-planar trans-linkage. FIG. 2 presents its 3-D structure, constructed by using a Cerius 2 Energy Minimization. As seen in the figure, the size of the OPV rod segment is about 2 nanometers in length and 0.5 nanometers in width, whereas the length of the extended PEO segment is about 6 nanometers. On the other hand, the worth noting part is that the sulfonate group is approximately tetrahedral-shaped. The sulfonate group cooperates with the OPV segment being co-planar and having a conjugate structure. Opposite to the soft PEO segment, the PPV segment is rigid. Therefore, the sulfonate group twists, to have a twist, between the PEO segment and the OPV segment of the amphiphilic conjugate molecule.

A second embodiment of the invention discloses a method for forming an amphiphilic molecule. In the method, a first starting material is provided, wherein the first starting material has the following structural formula: 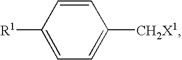
wherein the R¹ is an alkyl group and the X¹ is an element in group VIIA. A first substitution reaction is performed on the first starting material, to form a first alkene product having the following structural formula: 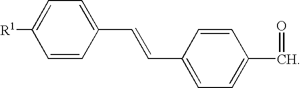
A second starting material is provided, wherein the second starting material has the following structural formula: 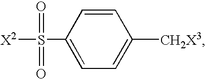
wherein the X2 and X3 are elements in group VIIA, and wherein the X2 is more reactive than the X3. A second substitution reaction is performed on the first alkene product, to form a second alkene product having the following structural formula 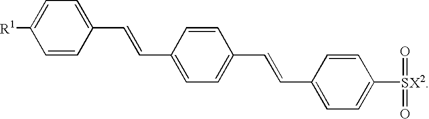
The poly(ethylene glycol) methyl ethers reacts with the second alkene product in an esterification reaction to form the amphiphilic molecule. The esterification reaction is catalyzed by pyridines. The amphiphilic molecule has the following structural formula: 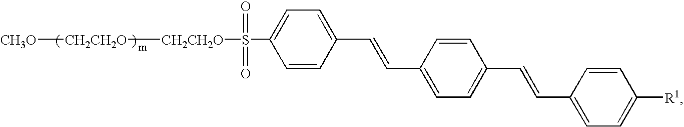
wherein the m≧1.

A third embodiment of the invention disclosed a method for forming an amphiphilic molecule. In the method, a first starting material having the following structural formula is provided: 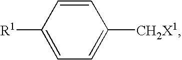
wherein the R¹ is an alkyl group and X1 is an element in group VIIA. A first trialkyl phosphate (P(OR²)₃) reacts with the first starting material in a first phosphonation reaction, to form a first intermediate having the following structural formula: 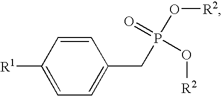
wherein the R² is an alkyl group. A Lewis base reacts with an aldehyde compound and the first intermediate in a first olefination reaction alkene, to form a first alkene product. The aldehyde compound and the first alkene product respectively have the following structural formulas: 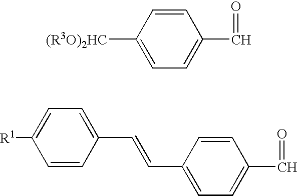
and wherein the R³ is an alkyl group, and wherein two of R¹, R² and R³ are identical or non-identical. A second starting material having the following structural formula: 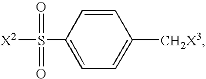
wherein the X² and X³ are elements in group VIIA, and X³ is more reactive than X².

In this embodiment, a second trialkyl phosphate (P(OR⁴)₃) reacts with the second starting material in a second phosphonation reaction, to form a second intermediate having the following structural formula: 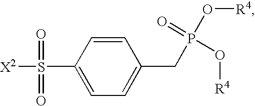
wherein the R⁴ is an alkyl group. The first intermediate and the second intermediate reacts with the Lewis base in a second olefination reaction alkene, to form a second alkene product having the following structural formula: 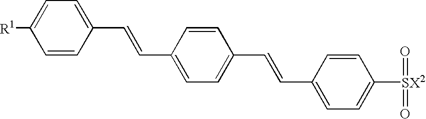
The Lewis base comprises a potassium t-butoxide (t-BuOK). The second alkene product reacts with poly(ethylene glycol)methyl ethers in an esterification reaction, to form the amphiphilic molecule. The esterification reaction is catalyzed by pyridines. The amphiphilic molecule has the following structural formula: 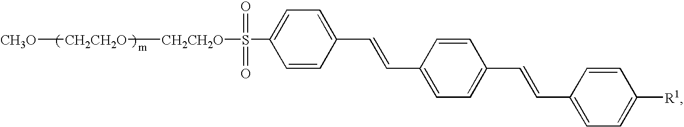
wherein the m≧1.

### EXAMPLE 2

### a. Synthesis of Dibutyl(4-tert-butylphenyl)methylphosphonate (1)

2 mL (10 mmol) 1-tert-butyl-4-(chloromethyl)benzene and 8 g (30 mmol) tributyl phosphate were placed in a flask with a magnetic stirring bar. The reaction mixture was stirred and refluxed at 160° C. under nitrogen for 12 h. After cooled to room temperature, the mixture was concentrated under reduce pressure at 200° C. to remove the residual tributyl phosphate. The resulting yellow oil was used for the next Horner-Wittig reaction without further purification. Yield: 80%.

¹H-NMR (500 MHz, CDCl3): δ/ppm: 7.08-7.32 (m, 4H), 3.99 (t, 4H), 3.77 (m, 2H), 1.57 (m, 4H), 1.45 (m, 4H), 1.34 (s, 9H), 0.91 (t, 6H).

### b. Synthesis of 4-(4-tert-butylstyryl)benzaldehyde (2)

A solution of 3.44 g (16 mmol) 4-(diethoxymethyl)benzaldehyde and 4.8 g (14 mmol) phosphonate (1) in 30 mL THF was prepared in a flask with a magnetic stirring bar and added dropwise with potassium t-butoxide solution in THF (1.8 g/60 mL). The mixture was stirred for 18 h at room temperature under nitrogen. Then, 50 mL of 37.5% HCl was added and the mixture was stirred for another 3 h. Afterwards, the reaction mixture was poured into a 500 mL water/ethanol (1:1 by volume) to allow a yellow crude product precipitated. The precipitates were filtered and washed with water and ethanol for several times. Recrystallization from dichloromethane/ethanol gave a pure yellow solid. Yield: 70%.

¹H-NMR (500 MHz, CDCl₃): δ/ppm: 9.97 (s, 1H), 7.84 (d, 2H), 7.62 (d, 2H), 7.47 (d, 2H), 7.40 (d, 2H), 7.23 (d, 1H), 7.10 (d, 1H), 1.33 (s, 9H).

### c. Synthesis of Dibutyl(4-sulfonyl chloride-pheny)methylphosphonate (3)

1 g (3.7 mmol) of 4-(bromo-methyl)benzene-1-sulfonyl chloride and 3 g (12 mmol) of tributyl phosphate were placed in a flask with a magnetic stirring bar. The reaction mixture was stirred and refluxed at 160° C. under nitrogen for 12 h. After cooled to room temperature, the mixture was concentrated under reduce pressure at 200° C. to remove the residual tributyl phosphate. The resulting brown oil was used for the next Horner-Wittig reaction without further purification. Yield: 90%.

¹H-NMR (500 MHz, CDCl3): δ/ppm: 7.03-7.40 (m, 4H), 4.00 (m, 4H), 3.03 (m, 2H), 1.61 (m, 4H), 1.59 (m, 4H), 0.89 (t, 6H).

### d. Synthesis of OPV Trimer End-Capped with Tert-Butyl and Sulfonyl Chloride Groups (4)

A solution of 0.25 g (1.9 mmol) aldehyde (2) and 0.5 g (2.5 mmol) phosphonate (3) in 30 mL THF was prepared in a flask with a magnetic stirring bar and added dropwise with potassium t-butoxide solution in THF (0.34 g/50 mL). The mixture was stirred for 18 h at room temperature under nitrogen. Then, 20 mL of 37.5% HCl was added and the mixture was stirred for another 3 h. Afterwards, the reaction mixture was poured into a 500 mL water to allow a dark yellow crude product precipitated. The precipitates were filtered and washed with water several times. Recrystallization from chloroform gave a dark brown solid. Yield: 65%.

¹H-NMR (500 MHz, CDCl3): δ/ppm: 8.15 (d, 2H), 7.59 (d, 2H), 7.43 (d, 2H), 7.38 (d, 2H), 7.21 (m, 4H), 7.11 (d, 4H), 1.31 (s, 9H).

### e. Synthesis of PEO₁₇-OPV₃ (5)

A solution of 0.3 g (0.4 mmol) poly(ethylene glycol) methyl ether (Mw=750 g/mol) and 0.22 g (0.5 mmol) OPV trimer (4) in 150 mL dry dichloromethane was prepared in a flask with a magnetic stirring bar under nitrogen atmosphere and added dropwise with pyridine (1 g in 20 mL dry dichloromethane). The mixture was stirred for 36 h at room temperature. Afterwards, the mixture was washed with 50 mL water three times and then dried over MgSO4. After removing the solvent with vacuum evaporator, dark viscous oil was obtained. For purification, the crude product was subjected to column chromatography using ethyl acetate as an eluent. A purified light yellow oil product was obtained with a yield of 25%.

¹H-NMR (500 MHz, CDCl3): δ/ppm: 7.05-8.05 (m, 16H), 3.61 (m, 68H), 3.33 (s, 3H), 1.33 (s, 9H).

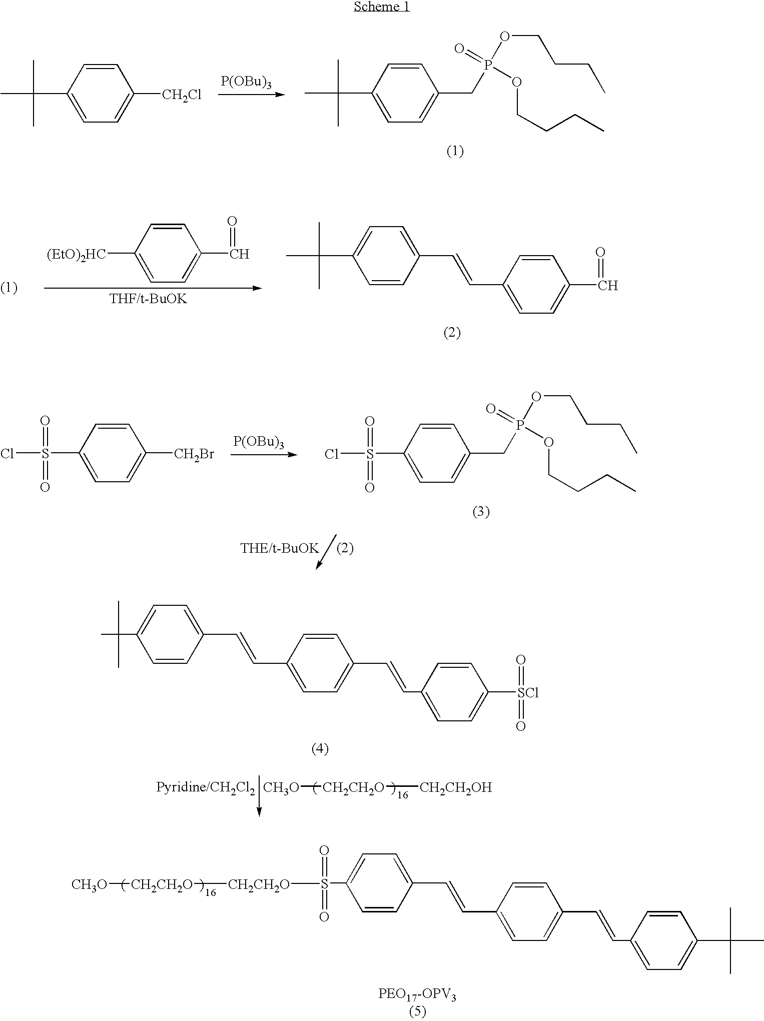

A fourth embodiment of the invention disclosed a primary supramolecular structure having a shape of a ring-like disk. The primary supramolecular structure comprises an amphiphilic conjugate molecule. The amphiphilic conjugate molecule comprises a hydrophobic segment, a hydrophilic segment and a linking group. The choices of the hydrophobic segment, the hydrophilic segment and the linking group, and those choices in the first embodiment, are substantially the same.

The linking group links the hydrophobic segment with the hydrophilic segment, and twists between the linked hydrophobic segment and the hydrophilic segment of the amphiphilic conjugate molecule. In a preferred example of the fourth embodiment, the amphiphilic conjugate molecule having the following structural formula: 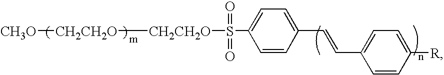
wherein m≧1, n≧1, and wherein the R is a cyclic alkyl group, a non-cyclic alkyl group, an aromatic group and a heterocyclic group. On the other hand, the shape of ring-like disk has a diameter of about 10 nanometers to 60 nanometers.

### EXAMPLE 3

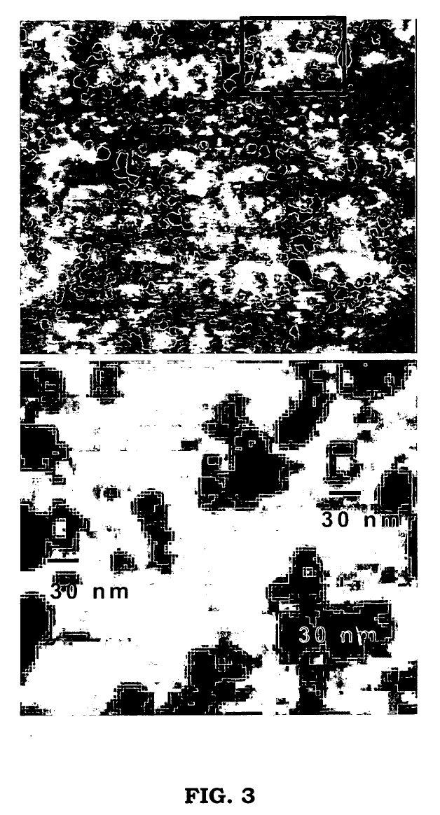

An amphiphilic conjugate molecule (abbreviated as “PEO₁₇—OPV₃”) having the following structural formula is provided: 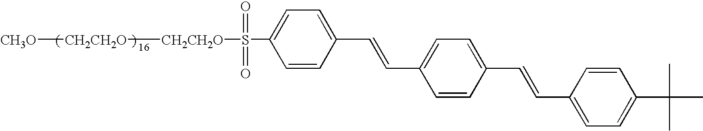
wherein the PEO segment has hydrophilicity, and the OPV segment has hydrophobility. Referring to FIG. 3, the primary supramolecular structure having a shape of a ring-like disk, formed with PEO₁₇—OPV₃ on a mica substrate, has a diameter of about 30 nanometers. Due to the soft nature of the PEO segment, the PEO₁₇—OPV₃ is also considered as a rod-coil molecule. Both of the amphiphilic and the rod-coil characteristics play major roles on the formation of self-assembling primary supramolecular structure.

Two factors are possibly involved for the formation of the shape of the ring-like disk: (1) The PEO₁₇—OPV₃ with a twist in between hydrophobic and hydrophilic segments render them to have only one side with the same curvature to proceed π-π stacking and; (2) There is very high interfacial tension between a hydrophilic substrate (for example, the mica substrate) and the hydrophobic OPV segment. To reduce the surface existence area, PEO₁₇—OPV₃ inclines, crosswise right-angle spreads and crimps the π-π stacking region.

A fifth embodiment of the invention discloses a method for forming a primary supramolecular structure having a shape of a ring-like disk. The method comprises the following steps. A solution of amphiphilic conjugate molecule is formed by suspending a plurality of the amphiphilic conjugate molecules in a solvent, wherein the solvent comprises water and tetrahydrofuran (THF), and wherein the volumetric ratio of the water and the tetrahydrofuran is about 1:1. The solution of amphiphilic conjugate molecule is at a concentration greater than about 10⁻⁵ M. A precipitating step is performed to deposit the solution of amphiphilic conjugate molecule on a hydrophilic substrate. The hydrophilic substrate is first maintained horizontally, so that the amphiphilic conjugate molecules interact with each other in the solution of amphiphilic conjugate molecule. The first maintaining step is performed for more than 36 hours. The solution of amphiphilic conjugate molecule is removed from the hydrophilic substrate, with a dropper. The hydrophilic substrate second is maintained horizontally. A thermal annealing step is performed, to form the primary supramolecular structure on the hydrophilic substrate. The thermal annealing step is performed at a temperature greater than about 100° C., and is performed in a vacuum of about 10⁻³ torrs.

A sixth embodiment of the invention disclosed a secondary supramolecular structure having a shape of ring-like disk. The secondary supramolecular structure comprises a plurality of amphiphilic conjugate molecules and a plurality of oxides, wherein the oxides are metal oxides or non-metal oxides. Therefore, the secondary supramolecular structure is a hybrid supramolecular structure. Each of the amphiphilic conjugate molecules comprises a hydrophobic segment being π-conjugated and having at least two aromatic structures, and further comprises a hydrophilic segment and a linking group. The linking group links the hydrophobic segment with the hydrophilic segment and twists between the linked hydrophobic segment and the hydrophilic segment of the amphiphilic conjugate molecule. On the other hand, the oxides are between the hydrophilic segments of the amphiphilic conjugate molecules. A recipe to form the oxides comprises one element selected from the group consisting of Li, Na, K, Rb, Cs, Be, Mg, Ca, Sr, Ba, Ti, Te, Cr, Cu, Er, Fe, Ta, V, Zn, Zr, P, B, Al, Si, Ge, Sn and Pb.

In the sixth embodiment, the choices of the hydrophobic segment, the hydrophilic segment and the linking group, and those choices in the first embodiment, are substantially the same. Additionally, a preferred example of the sixth embodiment, the amphiphilic conjugate molecule has the following structural formula: 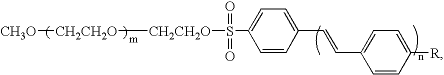
wherein m≧1, n≧1, and wherein the R is a cyclic alkyl group, a non-cyclic alkyl group, an aromatic group and a heterocyclic group. The shape of ring-like disk has a diameter of about 100 nanometers to about 300 nanometers.

### EXAMPLE 4

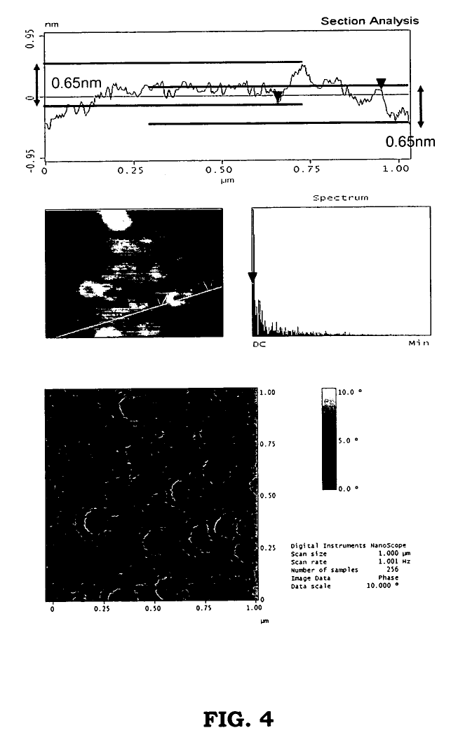

An amphiphilic conjugate molecule (abbreviated as “PEO₁₇—OPV₃”) having the following structural formula is provided: 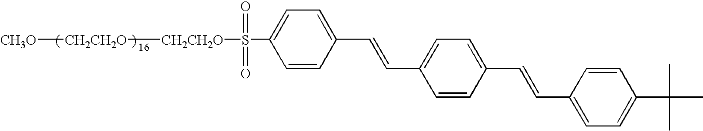
wherein the PEO segment has a hydrophilicity, and the OPV segment has a hydrophobility. Referring to FIG. 4, the supramolecular structure having a shape of a ring-like disk, formed with PEO₁₇—OPV₃/Silica hybrid on a mica substrate, has a diameter of about 150 nanometer and a thickness of about 0.65 nanometer.

The secondary supramolecular structure (hybrid supramolecular structure) comprises may be formed of silicates and amphiphilic conjugate molecules. The molar composition of the silicates and the amphiphilic conjugate molecules are about 1.0:0.19. Although the secondary supramolecular structure of PEO₁₇—OPV₃/Silica hybrid is approximately similar to that prepared from the neat PEO₁₇—OPV₃ solution, the diameter of ˜150 nm is much larger and more uniform. Notably, the thickness is roughly equal to the width of co-planar configuration of an OPV₃ rod segment (see FIG. 2). This indicates that amphiphilic PEO₁₇—OPV₃ molecules play as a template for the formation of secondary supramolecular structure of PEO₁₇—OPV₃/Silica hybrid.

A seventh embodiment of the invention discloses a method for forming a secondary supramolecular structure having a shape of a ring-like disk. A solution of salt precursor is formed by mixing ethanol, water, hydrochloric acid and alkoxides, wherein the alkoxides are metal alkoxides or non-metal alkoxides. The salt precursor has a pH value of about 2 to about 4. A solution of amphiphilic conjugate molecule is formed by suspending a plurality of the amphiphilic conjugate molecule in a solvent. The solvent comprises tetrahydrofuran(THF). Thereafter, a mixture is formed by mixing the solution of salt precursor with the solution of amphiphilic conjugate molecule. The mixture is then heated to about 40 to 90 degrees Celsius, preferably to about 60 degrees Celsius for about 90 minutes.

In the seventh embodiment, the mixture is first diluted by ethanol and water, to form a product solution, after the mixture is heated. The molar ratio of the alkoxides, the solvent, the water, the hydrochloric acid, the amphiphilic conjugate molecule and the ethanol is about 1.0:77:69:0.13:0.19:51.9 in the product solution. The product solution is second diluted by a solvent and water, to form a solution for deposition. The solvent, the water and the product solution have volumetric ratio of about 1:3:6 in the solution for deposition. The solution for deposition is deposited on the hydrophilic substrate. The hydrophilic substrate is first maintained horizontally. The amphiphilic conjugate molecules interact with each other in the solution of amphiphilic conjugate molecule. The hydrophilic substrate is first maintained horizontally for more than about 36 hours. Thereafter, a removing step is performed to remove the solution for deposition from the hydrophilic substrate. The hydrophilic substrate is second maintained horizontally. A thermal annealing step is performed to form the supramolecular structure on the hydrophilic substrate. Preferably, the thermal annealing step is performed at a temperature greater than about 100° C., and is performed in a vacuum of about 10⁻³ torrs.

An eighth embodiment of the invention discloses a recipe for forming a secondary supramolecule having a shape of ring-like disk. The recipe comprises a plurality of alkoxides, wherein the alkoxides are metal alkoxides or non-metal alkoxides. The alkoxides comprise one element selected from the group consisting of Li, Na, K, Rb, Cs, Be, Mg, Ca, Sr, Ba, Ti, Te, Cr, Cu, Er, Fe, Ta, V, Zn, Zr, P, B, Al, Si, Ge, Sn and Pb. The recipe further comprises an acid solution and an amphiphilic conjugate molecule. The amphiphilic conjugate molecule comprises a hydrophobic segment being π-conjugated, a hydrophilic segment and a linking group. The choices of the hydrophobic segment, the hydrophilic segment and the linking group, and those choices in the first embodiment, are substantially the same. In addition, linking group, linking the hydrophobic segment with the hydrophilic segment, and twisting between the linked hydrophobic segment and the hydrophilic segment of the amphiphilic conjugate molecule. The recipe, for forming a secondary supramolecule having a shape of ring-like disk, has a pH value of about 2 to about 4. On the other hand, in a preferred example of the eighth embodiment, the amphiphilic conjugate molecule having the following structural formula: 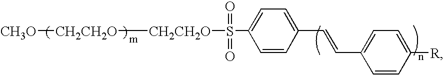
wherein m≧1, n≧1, and wherein the R is a cyclic alkyl group, a non-cyclic alkyl group, an aromatic group and a heterocyclic group.

According to the embodiments of the invention, an object of the invention is to provide an amphiphilic conjugate molecule. The amphiphilic conjugate molecule comprises a hydrophobic segment being π-conjugated, a hydrophilic segment and a linking group. The hydrophobic segment has at least two aromatic structures. The linking group links the hydrophobic segment with the hydrophilic segment. The linking group twists, to have a twist, between the linked hydrophobic segment and the hydrophilic segment of the amphiphilic conjugate molecule.

According to the embodiments of the invention, another object of the invention is to provide an amphiphilic conjugate molecule with a twist in between hydrophobic OPV segments and hydrophilic PEO segments render them to have only one side with the same curvature to proceed π-π stacking.

Additionally, the invention provides a hydrophilic substrate and a high interfacial tension between the hydrophilic substrate and the hydrophobic OPV segments. The high interfacial tension causes a π-π stacking region to curl in order to reduce the interfacial area. Thus, thermodynamically, the packing region of hydrophobic OPV segments prefers to form a ring structure unless the adhesion strength of hydrophilic PEO segments to the hydrophilic substrate is able to overcome this tendency. Besides, packing under the same curvature would favor formation of a shape of ring-like disk, so as to form the primary supramolecular structure.

It is further object of the invention to provide a secondary supramolecular structure (hybrid supramolecular structure). The secondary supramolecular structure, having a shape of ring-like disk, is formed of a plurality of metal or non-metal alkoxides and amphiphilic conjugate molecules. The diameter of the secondary supramolecular structure is much larger than the diameter of the primary supramolecular structure. The sizes between different secondary supramolecular structures are more uniform than the sizes of the different primary supramolecular structures.

To summarize, the invention discloses two kinds of supramolecular structures formed by amphiphilic conjugate molecules. The primary supramolecular structure has a shape of ring-like disk. The shape of ring-like disk has a diameter of about 10 nanometers to about 60 nanometers. Additionally, the invention also discloses a secondary supramolecular structure (hybrid supramolecular structure) having a shape of ring-like disk. The secondary supramolecular structure is formed of a plurality of metal or non-metal alkoxides and amphiphilic conjugate molecules. The shape of ring-like disk has a diameter of about 100 nanometer to about 300 nanometer.

Obviously, according to the detailed description of the above embodiments, this invention possibly has many revisions and the difference. There is therefore a need to understand the invention according to the appended claims. While the invention has been described by way of example and in terms of the preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. An amphiphilic conjugate molecule comprising:
a hydrophobic segment being a-conjugated, the hydrophobic segment having at least two aromatic structures;
a hydrophilic segment; and
a linking group, linking the hydrophobic segment with the hydrophilic segment, and twisting between the linked hydrophobic segment and the hydrophilic segment of the amphiphilic conjugate molecule.

2. The amphiphilic conjugate molecule of claim 1, wherein the hydrophobic segment comprises one selected from the group consisting of 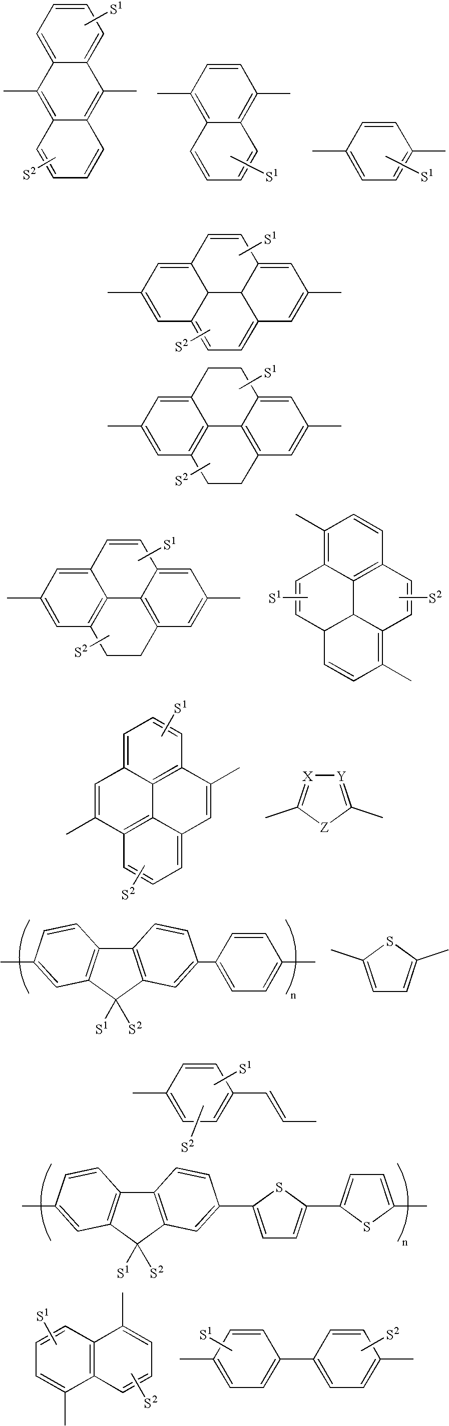 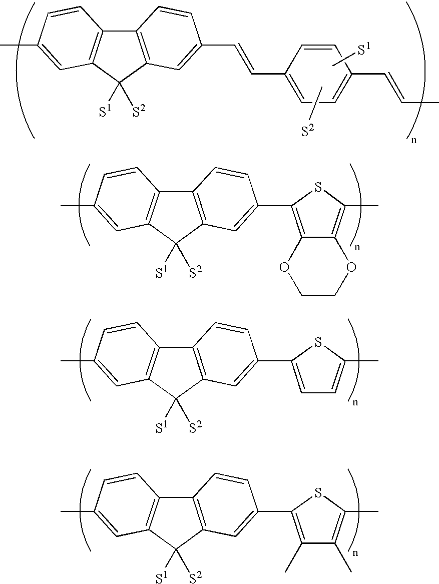
and the combination thereof, wherein two of the S¹, S², S³, S⁴ are identical or non-identical, and wherein the S1, S2, S3, S4 comprise one selected from the group consisting of a hydrogen atom, alkyl group, alkoxy group, cyclic alkyl group, aromatic group, heterocyclic group and the combination thereof, and wherein the X and Y comprise one selected from the group consisting of CS1, N and the combination thereof, and wherein the Z one selected from the group consisting of —O—, —S—, —NS1-, —CS1S2-, —CS1=CS1-, —CS1=N— and the combination thereof, and wherein the Ph is a phenyl group, and wherein the Ar is an aromatic group.

3. The amphiphilic conjugate molecule of claim 1, wherein the hydrophilic segment comprises one selected from the group consisting of polyalkylene glycol, polyalkylene glycol monoalkyl ether and their derivatives, wherein monoalkyl is selected from the group consisting of CH₃, C₂H₅, C₃H₇, and C₄H₉.

4. The amphiphilic conjugate molecule of claim 1, wherein the linking group comprises one selected from the group consisting of —CH₂—, —CR₂— and 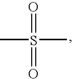
and wherein the R is an alkyl group.

5. The amphiphilic conjugate molecule of claim 1, wherein the amphiphilic conjugate molecule having the following structural formula: 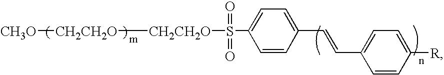
wherein m≦1, n≦1, and wherein the R is a cyclic alkyl group, a non-cyclic alkyl group, an aromatic group and a heterocyclic group.

6. A supramolecular structure having a shape of a ring-like disk, the supramolecular structure comprising an amphiphilic conjugate molecule, the amphiphilic conjugate molecule comprising:
a hydrophobic segment being g-conjugated, the hydrophobic segment having at least two aromatic structures;
a hydrophilic segment; and
a linking group, linking the hydrophobic segment with the hydrophilic segment, and twisting between the linked hydrophobic segment and the hydrophilic segment of the amphiphilic conjugate molecule.

7. The supramolecular structure of claim 11, wherein the hydrophobic segment comprises one selected from the group consisting of 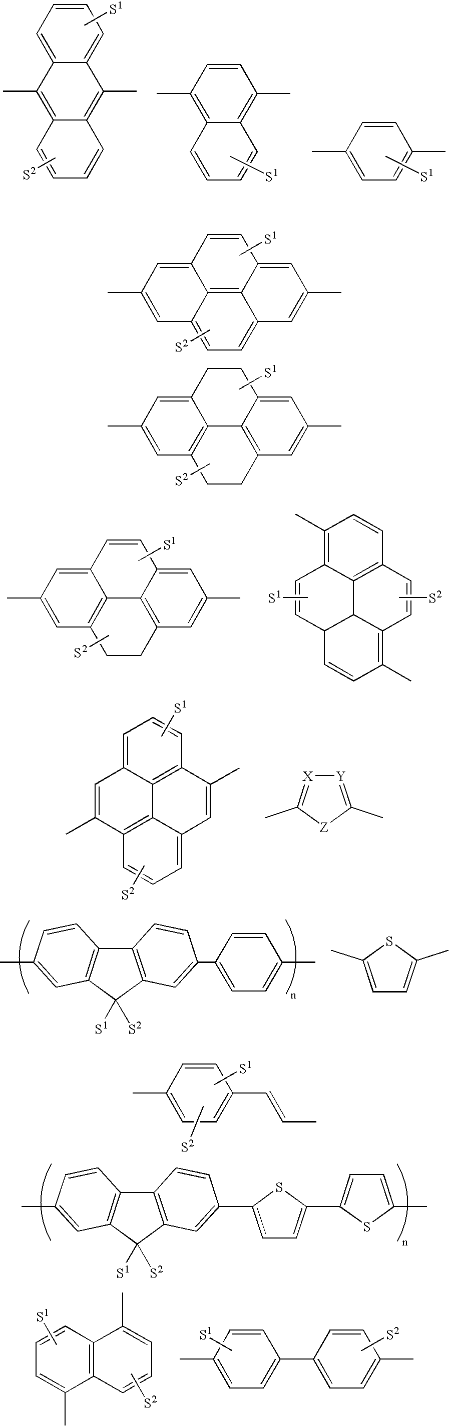 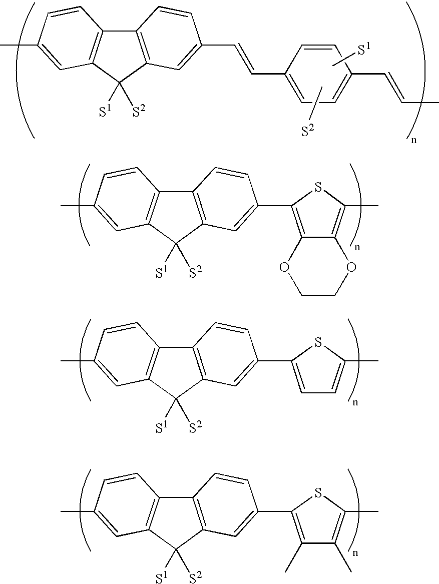
and the combination thereof, wherein two of the S¹, S², S³, S⁴ are identical or non-identical, and wherein the S¹, S², S³, S⁴ comprises one selected from the group consisting of a hydrogen atom, alkyl group, alkoxy group, cyclic alkyl group, aromatic group, heterocyclic group and the combination thereof, and wherein the X and Y comprise one selected from the group consisting of CS1, N and the combination thereof, and wherein the Z one selected from the group consisting of —O—, —S—, —NS1-, —CS1S2-, —CS1=CS1-, —CS1=N— and the combination thereof, and wherein the Ph is a phenyl group, and wherein the Ar is an aromatic group.

8. The supramolecular structure of claim 6, wherein the hydrophilic segment comprises one selected from the group consisting of polyalkylene glycol, polyalkylene glycol monoalkyl ether and their derivatives, wherein monoalkyl is selected from the group consisting of CH₃, C₂H₅, C₃H₇, and C₄H₉.

9. The supramolecular structure of claim 6, wherein the linking group comprises one selected from the group consisting of —CH₂—, —CR₂— and 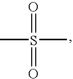
and wherein the R is an alkyl group.

10. The supramolecular structure of claim 6, wherein the amphiphilic conjugate molecule having the following structural formula: 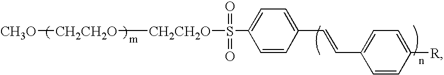
wherein m≦1, n≦1, and wherein the R is a cyclic alkyl group, a non-cyclic alkyl group, an aromatic group and a heterocyclic group.

11. The supramolecular structure of claim 6, wherein the shape of ring-like disk has a diameter of about 10 nanometers to 60 nanometers.

12. The supramolecular structure of claim 6, wherein the amphiphilic conjugate molecule has the following structural formula: 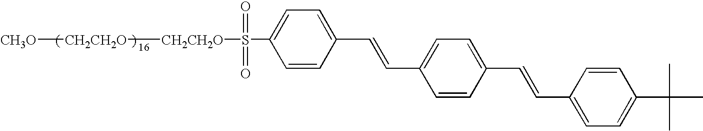
and the shape of ring-like disk has a diameter of about 30 nanometers.

13. The supramolecular structure of claim 6, wherein the supramolecular structure is formed by the following steps:
forming a solution of amphiphilic conjugate molecule by suspending a plurality of the amphiphilic conjugate molecules in a solvent;
performing a precipitating step to deposit the solution of amphiphilic conjugate molecule on a hydrophilic substrate;
first maintaining the hydrophilic substrate horizontally;
interacting the amphiphilic conjugate molecules with each other in the solution of amphiphilic conjugate molecule;
removing the solution of amphiphilic conjugate molecule from the hydrophilic substrate;
second maintaining the hydrophilic substrate horizontally; and
performing a thermal annealing step, to form the supramolecular structure on the hydrophilic substrate.

14. The supramolecular structure of claim 13 wherein the solvent comprises water and tetrahydrofuran (THF), and wherein the volumetric ratio of the water and the tetrahydrofuran is about 1:1.

15. The supramolecular structure of claim 13, wherein the solution of amphiphilic conjugate molecule is at a concentration greater than about 10⁻⁵ M.

16. The supramolecular structure of claim 13, wherein the first maintaining step is performed for more than 36 hours.

17. The supramolecular structure of claim 13, wherein the thermal annealing step is performed at a temperature greater than about 100° C.

18. The supramolecular structure of claim 13, wherein the thermal annealing step is performed in a vacuum of about 10⁻³ torrs.

19. A supramolecular structure having a shape of ring-like disk, the supramolecular structure comprising:
a plurality of amphiphilic conjugate molecules, each of the amphiphilic conjugate molecules comprising a hydrophobic segment being π-conjugated and having at least two aromatic structures, and further comprising a hydrophilic segment and a linking group linking the hydrophobic segment with the hydrophilic segment and twisting between the linked hydrophobic segment and the hydrophilic segment of the amphiphilic conjugate molecule; and
a plurality of oxides between the hydrophilic segments of the amphiphilic conjugate molecules, wherein the oxides are metal oxides or non-metal oxides.

20. The supramolecular structure of claim 19, wherein a recipe to form the oxides comprises one element selected from the group consisting of Li, Na, K, Rb, Cs, Be, Mg, Ca, Sr, Ba, Ti, Te, Cr, Cu, Er, Fe, Ta, V, Zn, Zr, P, B, Al, Si, Ge, Sn and Pb.

21. The supramolecular structure of claim 19, wherein the hydrophobic segment comprises one selected from the group consisting of 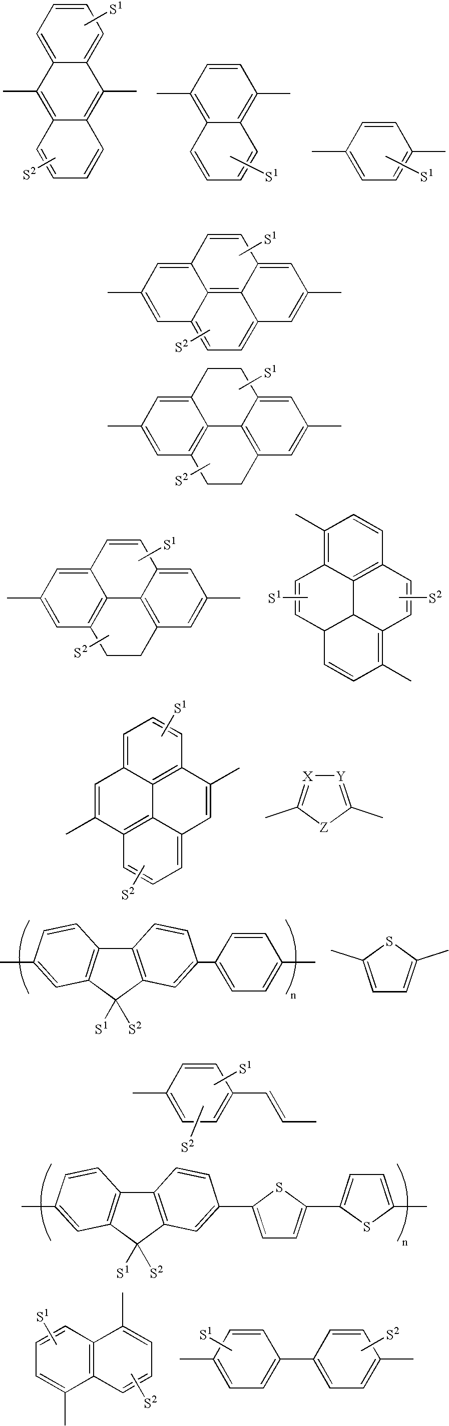 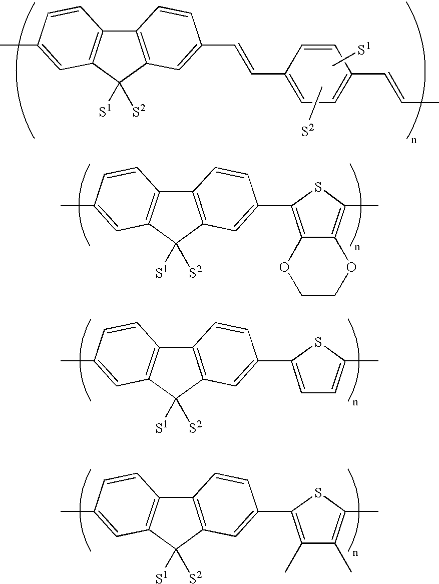
and the combination thereof, wherein two of the S¹, S², S³, S⁴ are identical or non-identical, and wherein the S¹, S², S³, S⁴ comprises one selected from the group consisting of a hydrogen atom, alkyl group, alkoxy group, cyclic alkyl group, aromatic group, heterocyclic group and the combination thereof, and wherein the X and Y comprise one selected from the group consisting of CS1, N and the combination thereof, and wherein the Z selects from the group consisting of —O—, —S—, —NS1-, —CS1S2-, —CS1=CS1-, —CS1=N— and the combination thereof, and wherein the Ph is a phenyl group, and wherein the Ar is an aromatic group.

22. The supramolecular structure of claim 19, wherein the hydrophilic segment comprises one selected from the group consisting of polyalkylene glycol, polyalkylene glycol monoalkyl ether and their derivatives, wherein monoalkyl is selected from the group consisting of CH₃, C₂H₅, C₃H₇, and C₄H₉.

23. The supramolecular structure of claim 19, wherein the linking group comprises one selected from the group consisting of —CH₂—, —CR₂— and 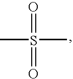
and wherein the R is an alkyl group.

24. The supramolecular structure of claim 19, wherein the amphiphilic conjugate molecule having the following structural formula: 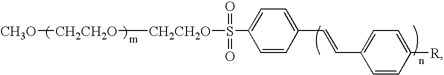
wherein m≦1, n≦1, and wherein the R is a cyclic alkyl group, a non-cyclic alkyl group, an aromatic group and a heterocyclic group.

25. The supramolecular structure of claim 19, wherein the shape of ring-like disk has a diameter of about 100 nanometers to about 300 nanometers.

26. The supramolecular structure of claim 19, wherein the amphiphilic conjugate molecule has the following structural formula: 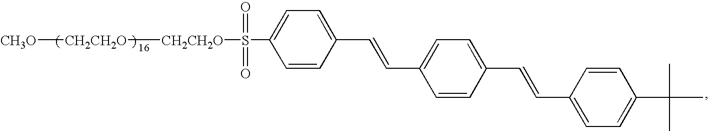
and the shape of ring-like disk has a diameter of about 150 nanometers and has a thickness of about 0.65 nanometers.

27. The supramolecular structure of claim 19, wherein the supramolecular structure is formed by the following steps:
forming a solution of salt precursor by mixing ethanol, water, hydrochloric acid and alkoxides, wherein the alkoxides are metal alkoxides or non-metal alkoxides;
forming a solution of amphiphilic conjugate molecule by suspending a plurality of the amphiphilic conjugate molecule in a solvent;
forming a mixture by mixing the solution of salt precursor with the solution of amphiphilic conjugate molecule;
heating the mixture;
first diluting the mixture by ethanol and water, to form a product solution;
second diluting the product solution by a solvent and water, to form a solution for deposition;
depositing the solution for deposition on the hydrophilic substrate;
first maintaining the hydrophilic substrate horizontally;
interacting the amphiphilic conjugate molecules with each other in the solution of amphiphilic conjugate molecule;
removing the solution for deposition from the hydrophilic substrate;
second maintaining the hydrophilic substrate horizontally; and
performing a thermal annealing step, to form the supramolecular structure on the hydrophilic substrate.

28. The supramolecular structure of claim 27, wherein the salt precursor has a pH value of about 2 to about 4.

29. The supramolecular structure of claim 27, wherein the solvent comprises tetrahydrofuran (THF).

30. The supramolecular structure of claim 27, wherein the mixture is heated to about 40 to 90 degrees Celsius.

31. The supramolecular structure of claim 27, wherein the molar ratio of the alkoxides, the solvent, the water, the hydrochloric acid, the amphiphilic conjugate molecule and the ethanol is about 1.0:77:69:0.13:0.19:51.9 in the product solution.

32. The supramolecular structure of claim 27, wherein the solvent, the water and the product solution have volumetric ratio of about 1:3:6 in the solution for deposition.

33. The supramolecular structure of claim 27, wherein the hydrophilic substrate is first maintained horizontally for more than about 36 hours.

34. The supramolecular structure of claim 27, wherein the thermal annealing step is performed at a temperature greater than about 100° C.

35. The supramolecular structure of claim 27, wherein the thermal annealing step is performed in a vacuum of about 10⁻³ torrs.

36. A recipe for forming a hybrid supramolecule having a shape of ring-like disk, the recipe comprising:
a plurality of alkoxides, wherein the alkoxides are metal alkoxides or non-metal alkoxides;
an acid solution; and
an amphiphilic conjugate molecule comprising:
a hydrophobic segment being a-conjugated, the hydrophobic segment having at least two aromatic structures;
a hydrophilic segment; and
a linking group, linking the hydrophobic segment with the hydrophilic segment, and twisting between the linked hydrophobic segment and the hydrophilic segment of the amphiphilic conjugate molecule.

37. The recipe of claim 36, wherein the alkoxides comprises one element selected from the group consisting of Li, Na, K, Rb, Cs, Be, Mg, Ca, Sr, Ba, Ti, Te, Cr, Cu, Er, Fe, Ta, V, Zn, Zr, P, B, Al, Si, Ge, Sn and Pb.

38. The recipe of claim 36, the recipe has a pH value of about 2 to about 4.

39. The recipe of claim 36, wherein the hydrophobic segment comprises one selected from the group consisting of 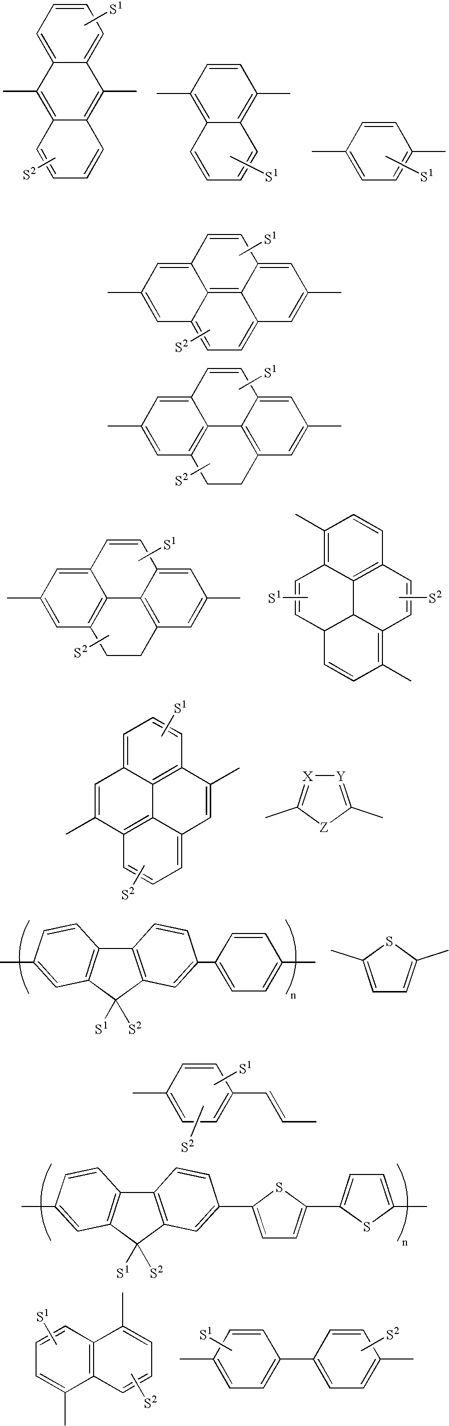 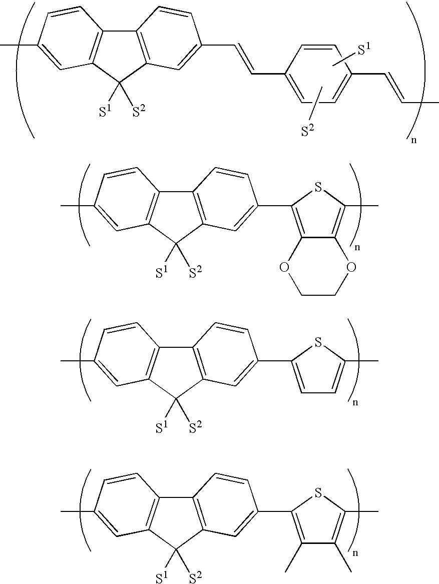
and the combination thereof, wherein two of the S¹, S², S³, S⁴ are identical or non-identical, and wherein the S1, S2, S3, S4 comprise one selected from the group consisting of a hydrogen atom, alkyl group, alkoxy group, cyclic alkyl group, aromatic group, heterocyclic group and the combination thereof, and wherein the X and Y comprise one selected from the group consisting of CS1, N and the combination thereof, and wherein the Z one selected from the group consisting of —O—, —S—, —NS1—, —CS1S2-, —CS1=CS1-, —CS1=N— and the combination thereof, and wherein the Ph is a phenyl group, and wherein the Ar is an aromatic group.

40. The recipe of claim 36, wherein the hydrophilic segment comprises one selected from the group consisting of polyalkylene glycol, polyalkylene glycol monoalkyl ether and their derivatives, wherein monoalkyl is selected from the group consisting of CH₃, C₂H₅, C₃H₇, and C₄H₉.

41. The recipe of claim 36, wherein the linking group comprises one selected from the group consisting of —CH₂—, —CR₂— and 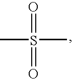
and wherein the R is an alkyl group.

42. The recipe of claim 36, wherein the amphiphilic conjugate molecule having the following structural formula: 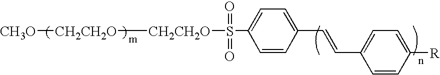
wherein m≦1, n≦1, and wherein the R is a cyclic alkyl group, a non-cyclic alkyl group, an aromatic group and a heterocyclic group.
